(19) United States
(12) Patent Application Publication
(10) Pub. No.: US 2026/0275427 A1
(43) Pub. Date: Sep. 17, 2026

(54) Rapid Viability Analysis

(71) Applicant: BATTELLE MEMORIAL INSTITUTE, Columbus, OH (US)

(72) Inventors: Rachel Spurbeck, Columbus, OH (US); Lindsay Catlin, Columbus, OH (US); Anna Adetona, Columbus, OH (US)

(21) Appl. No.: 19/562,675

(22) Filed: Mar. 10, 2026

Related U.S. Application Data
(60) Provisional application No. 63/770,809, filed on Mar. 12, 2025.

(51) Int. Cl. C12Q 1/689; C12Q 1/6895; C12Q 1/70

(52) U.S. Cl. CPC C12Q 1/689; C12Q 1/6895; C12Q 1/70; C12Q 2600/156

(57) ABSTRACT

Methods are provided for detecting and identifying viable microorganisms in a biological sample. Advantageously, the current method allows for the simultaneous screening of a sample for multiple pathogens selected from bacterial, fungal and viral pathogens.

### CROSS REFERENCE TO RELATED APPLICATIONS

This application claims priority to U.S. Provisional Patent Application No. 63/770,809 filed on Mar. 12, 2025, the disclosure of which is expressly incorporated herein.

### GOVERNMENT RIGHTS

This invention was made with government support under HR001124C0482, awarded by the Defense Advanced Research Projects Agency (DARPA), and MTEC-22-02-MPAI-050 awarded by the US Army. The government has certain rights in the invention.

### BACKGROUND

The most fundamental physiological state of eukaryotic, prokaryotic, fungal, and archaeal cells and viruses is their viability, defined here as the capacity to form progeny. For microbiologists, virologists, ecologists, pathobiologists, metagenomicists, food or water safety analysts, epidemiologists, infectious-disease clinicians, the observation of a viable microorganism in a sample means something entirely different from the observation of a dead one. Dead bacteria were reported to account for 32% of the total bacteria in fresh feces and their DNA can persist for up to a year in a laboratory environment. Despite its importance, distinguishing between dead and viable microbes present in a sample remains extremely challenging by current microbiological methods.

The current state of the art for viability testing is to use rapid viability qPCR which looks at one organism at a time, comparing the gene copy number before and after exposure of the sample comprising the organism to conditions favorable for growth. Other known methods, such as live dead staining only work for bacteria or fungi, and again are not suitable for characterizing the viability of multiple microbes simultaneously.

### SUMMARY

As disclosed herein a method of detecting viable microorganisms in a sample is provided that is based on a quantitative comparison of nucleic acids recovered from a sample at an initial timepoint and a later timepoint. The later timepoint represents a time after the initial sample is subject to conditions favorable for growth and replication of the microorganism. This method enables rapid viability testing for all organisms in a sample capable of growth under the cultivation conditions chosen.

In one embodiment nucleic acids isolated from organisms in a sample (blood, saliva, wastewater, water, soil, swab, food, etc) are analyzed by quantitative PCR/RT-qPCR or by DNA or RNA sequencing at an initial time zero (T₀), including for example, at the time of sampling from the environment. The information provided by the analysis of the T₀ nucleic acids is then compared to a similar analysis conducted on nucleic acids isolated from a similarly sourced sample after growth for a set amount of time in different cultivation media (including for example, with or without co-culture with a pre-selected host cell to enable viral growth). The genome copy number for each organism is normalized to an artificial spike-in control and the normalized genome copies for each organism is compared before and after growth to determine the growth or increase in genome copies over time as an indicator of microorganism viability.

In accordance with one embodiment, the identification and viability of multiple bacterial strains present in a sample is determined based on analysis of 16S rRNA. In accordance with one embodiment the identification and viability of multiple fungal strains present in a sample is determined based on analysis of internal transcribed spacer (ITS) sequences (representing a non-coding region of fungal ribosomal RNA (rRNA) genes). For viruses, whole genome sequencing or RNA sequencing can be employed dependent on if the virus of interest comprises a DNA or RNA genome.

The method of the present disclosure overcomes disadvantages associated with metagenomics where the analysis cannot determine if the organisms identified by sequencing is alive or dead. In accordance with one embodiment the method disclosed herein enables identification of viable pathogens present in a sample by comparing the genome copy number of selected pathogens before and after cultivation of the sample, or alternatively by comparing the copy number of one or more individual genes (e.g, 16S rRNA or ITS sequences) before and after cultivation of the sample.

In accordance with one embodiment a method of detecting and identifying viable microorganisms in a sample is provided, optionally wherein the sample is screened for viable pathogenic organisms selected from the group consisting of bacterial, fungal, protist or viral pathogens. In one embodiment the method comprises the steps of isolating a first population of nucleic acids from an initial sample;
exposing a sample (either a portion of the initial sample or a separate sample that was isolated from the same source) to conditions favorable to the growth and replication of microorganisms, for a predetermined length of time;
isolating a second population of nucleic acids from the sample that was cultured for the predetermined length of time; and comparing the copy number of the microorganism genome, or the gene copy number of an individual gene of the microorganism, in the second population of nucleic acids relative to the first population of nucleic acids, wherein an increase in copy number in the second population of nucleic acids relative to the first population of nucleic acids indicates the presence of viable microorganisms in the sample. In one embodiment, the sample is simultaneously screened for the presence of multiple viable organisms based on an analysis of the nucleic acid sequences that are increased in copy number after the culture step. In one embodiment the sample is simultaneously screened for the presence of bacterial and fungal organisms. In one embodiment the sample is simultaneously screened for the presence of bacterial and viral organisms. In one embodiment the sample is simultaneously screened for the presence of viral and fungal organisms. In embodiments where the sample is simultaneously screened for bacterial, fungal and/or viral organisms, the original sample may be further divided to allow optimal culturing conditions for each organism to be screened for viability.

In accordance with one embodiment the nucleic acids of the first and second population of nucleic acids are subjected to quantitative polymerase chain reaction (qPCR), optionally in the presence of a spike-in control. In one embodiment the spike-in control will comprise a mixture of sequences representative of fungi, bacteria, and viruses. In one embodiment the nucleic acids of the first and second population of nucleic acids are RNA molecules and the isolated RNA is subjected to reverse transcriptase quantitative polymerase chain reaction (RT-qPCR), optionally in the presence of a spike-in control.

In one embodiment the method simultaneously identifies multiple viable microorganisms present in a sample, including for example the presence of fungi, bacteria, viruses, and pathogenic species of such microorganisms. For example, qPCR or RT-qPCR can be conducted using PCR primers specific for bacterial 16S rRNA and primers specific for fungal ITS sequences to simultaneously detect pathogenic bacterial or fungal species.

In one embodiment the nucleic acids of the first and second population of nucleic acids are sequenced to detect and identify viable bacterial, protist, fungal and viral species present in a sample, wherein the first population of nucleic acids represents nucleic acids isolated from a sample at time T₀ and the second population of nucleic acids represents nucleic acid isolated from a sample at a later time (Tₓ), after the T₀ sample has been cultured under conditions suitable for microorganism growth and replication. A quantitative comparison of the sequenced nucleic acids of the first and second populations of nucleic acids, after normalization of the two populations of nucleic acids using a spike-in control, identifies microorganisms present in the sample. Furthermore, a detected increase in the copy number of the sequenced nucleic acids in the second population of nucleic acids relative to the sequenced nucleic acids in the first population of nucleic acids indicates the presence of viable microorganisms in the sample.

### BRIEF DESCRIPTION OF THE DRAWINGS

FIG. 1 provides a schematic representation of a viability sequencing method. Nucleic acid extracts are prepared from a sample comprising four target organisms (Org 1, Org 2, Org 3, and Org 4) at an initial time (T₀). Nucleic acid extracts are also prepared from a sample comprising four target organisms (Org 1, Org 2, Org 3, and Org 4) time (Tₓ) after the sample has been cultured for a predetermined length of time under conditions favorable for growth of the target organisms. Optionally, the extracted nucleic acids from the (T₀) and (Tₓ) samples are subject to a step of subtractive hybridization to remove abundant nucleic acids present in the nucleic acid extract and enhance detection of less abundant target sequences. Libraries of the extracted nucleic acids are prepared using equal volumes of the (T₀) and (Tₓ) nucleic acid samples. The samples are then analyzed by qPCR and normalized, followed by sequencing of at least some of the nucleic acids.

FIGS. 2A and 2B provide the process for screening for viable bacterial strains in a sample. FIG. 2A describes the general process and FIG. 2B shows the results of determining the viability of eleven different bacteria in a sample.

FIGS. 3A and 3B provide viability qRT-PCR plots. FIG. 3A is a 16S Amplification plot for B. subtilis on TSA after 24 hours demonstrates a viability factor of 6.16. FIG. 3B is an ITS Amplification plot for Candida albicans Malt Extract Broth at 48 hour growth demonstrates a viability factor of 5.72.

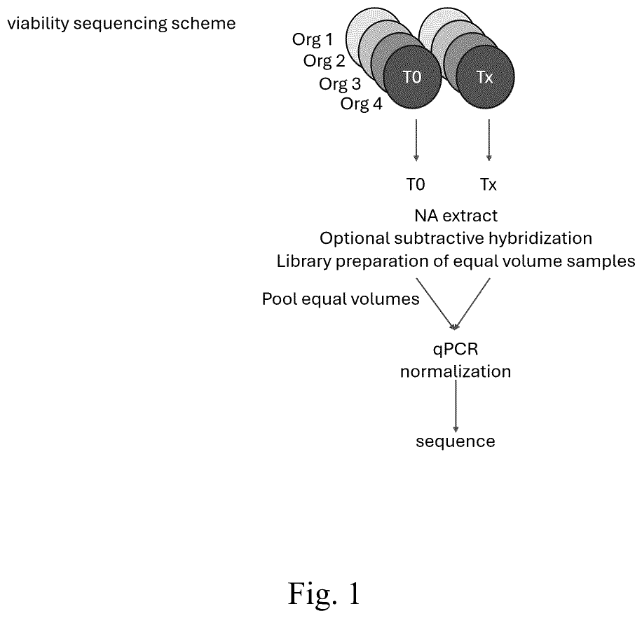

### DETAILED DESCRIPTION

### Definitions

As used herein the phrase “conditions favorable to the growth and replication of a microorganism” defines the combination of specific controlled environmental and nutritional factors needed to successfully grow and maintain a biological culture (e.g., bacterial culture) in a laboratory, including factors like temperature, pH level, nutrient composition, and oxygen concentration, that are carefully regulated to optimize the growth of the organism of interest.

As used herein the term “spike-in control” defines one or more exogenous nucleic acid sequences, not initially present in a sample, that are added to a sample to act as an internal standard. The spike-in control functions to confirm that the assay is functioning in a non-biased manner and can serves as a common reference between separate assays (e.g., PCR amplifications or RNA sequencing reactions conducted across multiple samples) to normalize the data produced by separate assays. The spike in control also enables comparison between samples with uneven sequencing depth. In one embodiment, the inclusion of a spike in control allows for the detection of, and normalization of differences in amplification rates between a T₀ and a Tₓ sample. For Example, by normalizing all data within a sample to a known quantity spike in control, if a sample had 1000000 reads at T0 and 1000000000 at T24, but the spike in control had 1000 reads of the T0 and 1000000 at T24, then a microbe that has 10 reads at T0 and 10000 reads at T24 would be equivalent meaning no growth in 24 hrs, whereas a different organism with 10 reads at T0 and 30000 reads at T24 in the same sequencing run would have grown and thus be identified as being viable.

In another embodiment the spike in control consists of a pool of synthetic transcripts that cover a range of concentrations. For example, there may be 1 molecule of A, 2 of B, 3 of E, and so on. The number of counts in an RNA-seq experiment should mirror the concentration range.

### EMBODIMENTS

The present disclosure is directed to methods for detecting and identifying viable microorganisms in a biological sample. Advantageously, the current method allows for the simultaneous screening of a sample for multiple pathogens selected from bacterial, fungal and viral pathogens. The method comprises analyzing the nucleic acid sequences isolated from a biological sample at an initial time (T₀) and then again at a pre-determined second timepoint (Tₓ) after the T₀ biological sample has been cultured under conditions favorable for growth and reproduction of the microorganisms present in the sample. In accordance with one embodiment an original sample is obtained and divided into two or more portions wherein nucleic acids are recovered from a first portion of the sample at time T₀ and the remaining portions of the initial sample are each separately subjected to culture conditions suitable for growth of one or more microorganisms to be detected. For detection of viral pathogens the culture conditions will include the presence of suitable host cells.

In accordance with one embodiment a method of screening for two or more viable microorganisms in a sample is provided, wherein the two or more microorganisms are independently selected from bacteria, fungi, protists or viruses. In one embodiment the method comprises
i) determining the concentration of a first microorganism nucleic acid and a second microorganism nucleic acid in a sample at a first timepoint;
ii) determining the concentration of said first microorganism nucleic acid and said second microorganism nucleic acid in said sample at a second timepoint, wherein the sample is exposed to conditions favorable for growth of microorganisms present in the sample during the time interval between said first timepoint and said second timepoint, wherein an increase in the concentration of said first microorganism nucleic acid or said second microorganism nucleic acid at the second timepoint relative to the first timepoint identifies the presence of a viable microorganism in the sample. In one embodiment the first microorganisms detected are bacterial organisms and the second microorganism detected are fungal species. In one embodiment the nucleic acids of a first portion of a sample are extracted at a first timepoint (T₀) and a second portion of the sample is cultured under conditions favorable for growth of one or more target microorganisms for a predetermined length of time (Tₓ) before extracting nucleic acids from the second portion of the sample. Equivalent volumes of the nucleic acid extracts isolated from the T₀ and Tₓ samples are then compared using analytical tools including qPCR, RTqPCR and next generation sequencing to identify whether there is a relative increase in nucleic acids in the Tₓ sample relative to the T₀ sample, thus indicating the presence of one or more viable microorganisms.

In accordance with one embodiment a method of screening for two or more groups of viable microorganisms in a sample is provided, wherein the two or more groups of microorganisms represent different classes of microorganisms selected from bacteria, fungi, protists or viruses. In one embodiment the method comprises
i) determining the concentration of a first class of microorganism nucleic acid and a second class of microorganism nucleic acid in a sample at a first timepoint;
ii) determining the concentration of said first class of microorganism nucleic acid and said second class of microorganism nucleic acid in said sample at a second timepoint, wherein the sample is divided and exposed to conditions favorable for growth of first and second classes of microorganisms present in the sample during the time interval between said first timepoint and said second timepoint, wherein an increase in the concentration of said first class microorganism nucleic acids or said second class of microorganism nucleic acids at the second timepoint relative to the first timepoint identifies the presence of a viable microorganism in the sample. In one embodiment the first class of microorganisms represents all the bacterial species present in the original sample, and the second class of microorganisms represents all the fungal species present in the original sample. In one embodiment the first class of microorganisms represents all the bacterial species present in the original sample, and the second class of microorganisms represents all the viral species present in the original sample. In one embodiment the first class of microorganisms represents all the viral species present in the original sample, and the second class of microorganisms represents all the fungal species present in the original sample.

In accordance with one embodiment the method simultaneously screens for the presence of viable bacterial and fungal species, wherein the first microorganism nucleic acid is a bacterial nucleic acid, and the second microorganism nucleic acid is fungal nucleic acid. In one embodiment the microorganism nucleic acid is DNA, and the genome copy number, or specific gene copy number, of the respective microorganisms is determined at both T₀ and at Tₓ. In one embodiment a universal bacterial qPCR is conducted on the first microorganism nucleic acid at both T₀ and at Tₓ, using primers targeting 16S rRNA, and a universal fungal qPCR is conducted on the second microorganism nucleic acid at both T₀ and at Tₓ, using primers targeting Internal Transcribed Spacer (ITS) regions to identify the viability of individual bacterial and fungal species in the sample.

In one embodiment the nucleic acids isolated from the microorganisms is RNA and the relative concentration of the RNAs present at both T₀ and at Tₓ is determined. In one embodiment the isolated RNA is analyzed by conducting a reverse transcriptase quantitative polymerase chain reaction (RT-qPCR) at both T₀ and at Tₓ, optionally followed by sequencing the resulting cDNAs.

In accordance with one embodiment an original sample is subdivided into separate portions wherein each sample portion is processed in a manner most suitable for detection and identification of a particular microorganism including those selected from the group of bacteria, fungi, protists or viral microorganisms. Depending on the type of microorganism to be identified, the nucleic acid isolated from the sample can be RNA or DNA. Specifically, when screening for biologically active RNA viruses, RNA is isolated from the sample. In addition, different sample portions can be cultured under different conditions optimized for the growth and reproduction of a particular microorganism selected from the group of bacteria, fungi, protists or viral microorganisms. Additionally, the length of time between T₀ and at Tₓ can be optimized based on the growth rate of the microorganism, with longer culture times required for fungi relative to bacterial and viral microorganisms. Typically, the time between said first timepoint and said second timepoint ranges from about 24 hours to 14 days. In one embodiment when screening for bacterial or viral microorganisms the time between said first timepoint and said second timepoint ranges from about 24 hours to 48 hours. In one embodiment, when screening for fungi, the time between said first timepoint and said second timepoint ranges from about 48 hours to 14 days.

In accordance with one embodiment the nucleic acids sequences isolated from the sample are sequenced either by using whole genome sequencing techniques or by targeted sequencing of specific genes (e.g. 16S rRNA or ITS genes). In one embodiment a method of detecting viable microorganisms in a sample is provided wherein the method comprises
isolating a first population of nucleic acids from a first portion of said sample;
exposing a second portion of said sample to conditions favorable to the growth and replication of microorganisms, for a predetermined length of time;
isolating a second population of nucleic acids from said second portion of said sample after said predetermined length of time, wherein the volume of the first population of nucleic acids and the second population of nucleic acids are equivalent when conducting further analysis of the extracted nucleic acids;
sequencing equivalent volumes of the nucleic acids isolated from said first and second populations of nucleic acids; and
conducting a quantitative comparison of the sequenced nucleic acids of the first and second populations of nucleic acids wherein an increase in the number of the sequenced nucleic acids in the second population of nucleic acids relative to the sequenced nucleic acids in the first population of nucleic acids indicates the presence of viable microorganisms in the sample. In one embodiment the genome copy number for each organism is normalized using an artificial spike-in control. Accordingly, in accordance with the present disclosure a sample can be analyzed simultaneously to assess the presence of viable microorganisms within a particular class of organism (e.g., different species of bacteria) or assess the presence of viable microorganisms from across difference classes of organisms, including fungi, bacteria, viruses, and other microorganisms present in a sample

In accordance with one embodiment the isolated first and second population of nucleic acids is DNA, wherein the isolated DNA of the first and second population of nucleic acids is sequenced using a whole genome library preparation method. The sequences are then analyzed to identify individual organism DNAs, as well as compare the relative copy numbers of the individual organism DNAs present in the first and second population of nucleic acids, wherein a detected increase in copy number in the second population of nucleic acids relative to the first population of nucleic acids identifies viable organisms present in the original sample. Alternatively, the sequences can be analyzed by read count mapping to a reference microorganism's genome, by counting how many sequences map to specific genomic features after they are aligned to the reference genome, providing a measure of their abundance.

In accordance with one embodiment the isolated first and second population of nucleic acids is RNA and the method of analyzing the nucleic acids in the isolated first and second population of nucleic acids comprises the steps of reverse transcribing the isolated RNA of the first and second populations of nucleic acid using a random hexamer to generate cDNA, and then sequencing the resulting cDNA through the use of library preparation for the appropriate sequencer. The sequences are then analyzed to identify individual organism cDNAs and as well as compare the relative copy numbers of the individual organism cDNAs produced from the first and second population of nucleic acids wherein a detected increase in copy number in the second population of nucleic acids relative to the first population of nucleic acids identifies viable organisms present in the original sample.

In another embodiment the total mapped sequence reads to the genome is compared between the first and second population of nucleic acids wherein a detected increase in total mapped reads in the second population of nucleic acids relative to the first population of nucleic acids identifies viable organisms present in the original sample. These methods allow quantification of RNA viruses, or any other organism that produces RNA in the original sample. In one embodiment when RNA is isolated from a sample, the isolated RNA can be treated with an RNAse-free DNAse to remove any contaminating DNA from the sample, preferably prior to the amplification or sequencing of the isolated RNA.

In accordance with one embodiment when the sample is screened to detect infectious viruses, the viruses are generally poorly represented due to the small genome size of viruses compared to bacteria, fungi, archaea, and eukaryotes. Therefore, in one embodiment, the nucleic acids isolated from a sample are first processed to deplete the samples of highly abundant or off target nucleic acids, such as ribosomal RNA and host eukaryotic RNA, to enable sequencing of the smaller genomes in a mixed sample. In one embodiment, the method of subtractive hybridization is utilized to remove rRNA from bacteria or fungi. Similarly, subtractive hybridization can be utilized on the viral samples to remove any cell line nucleic acids and enrich for vial nucleic acids. In one embodiment suitable for RNA viruses, host cell host cell RNA is removed from isolated nucleic acid of the sample by subtractive hybridization wherein DNA from the cell line is used as a probe for creating RNA: DNA duplexes prior to an RNase H treatment. RNaseH selectively degrades only RNA hybridized to DNA. Therefore, in a pure RNA sample, only RNA molecules that bind to a DNA probe will be degraded and removed. DNase I is then used to remove the probes and host cell DNA, and the RNA sample is depleted of high abundant RNAs such as host RNA or rRNA. Sequencing libraries can then be produced from the viral RNA enriched composition.

### EXEMPLARY EMBODIMENTS

In accordance with embodiment 1, a method of identifying viable microorganisms in a sample is provided wherein the method comprises
i) isolating nucleic acids from the sample and determining the concentration of a first microorganism nucleic acid and a second microorganism nucleic acid in a sample at a first timepoint;
ii) isolating nucleic acids from the sample and determining the concentration of said first microorganism nucleic acid and said second microorganism nucleic acid in said sample at a second timepoint after the first timepoint, wherein the sample is exposed to conditions favorable for growth of microorganisms present in the sample during the time interval between said first timepoint and said second timepoint, wherein an increase in the concentration of said first microorganism nucleic acid or said second microorganism nucleic acid at the second timepoint relative to the first timepoint identifies the presence of a viable microbe in the sample.

In accordance with embodiment 2 a method of detecting viable microorganisms in a sample is provided, wherein the method comprises
isolating a first population of nucleic acids from a first portion of said sample;
exposing a second portion of said sample to conditions favorable to the growth and replication of microorganisms, for a predetermined length of time;
isolating a second population of nucleic acids from said second portion of said sample after said predetermined length of time;
sequencing an equivalent volume of nucleic acids isolated from said first and second populations of nucleic acids; and
conducting a quantitative comparison of the sequenced nucleic acids of the first and second populations of nucleic acids wherein an increase in the number of the sequenced nucleic acids in the second population of nucleic acids relative to the sequenced nucleic acids in the first population of nucleic acids indicates the presence of viable microorganisms in the sample.

In accordance with embodiment 3, the method of embodiment 1 or 2 is provided wherein microorganisms screened for viability are organisms independently selected from bacterial, fungal, protists or viral species, optionally wherein the first microorganism is a bacterial organism, and the second microorganism is a viral organisms, or the first microorganism is a viral organism and the second microorganism is a fungal organism, or the first microorganism is a bacterial organism and the second microorganism is a fungal organism, wherein the original sample is divided into multiple portions to allow optimal culturing conditions for each organism to be screened for viability.

In accordance with embodiment 4, the method of any one of embodiments 1-3 is provided wherein the sample is simultaneously screened for the presence of two or more different species of microorganisms wherein a first and second microorganism
i) are organisms independently selected from bacterial, fungal and DNA virus species; or
ii) are both bacterial species; or
iii) are both fungal species;
   and the isolated nucleic acid is DNA.

In accordance with embodiment 5, the method of any one of embodiments 1-3 is provided wherein the sample is simultaneously screened for the presence of two or more different species of RNA viruses and the isolated nucleic acid is RNA, optionally wherein the concentration of said first microorganism nucleic acid and said second microorganism nucleic acid is measure by conducting a reverse transcriptase quantitative polymerase chain reaction (RT-qPCR).

In accordance with embodiment 6, the method of any one of embodiments 1~4 is provided wherein
i) the first microorganism and the second microorganism is a bacterial species and a universal bacterial qPCR is conducted on the DNA isolated from the sample, using primers targeting 16S rRNA encoding sequences; or
ii) the first microorganism and the second microorganism is a fungal species and a universal fungal qPCR is conducted on the DNA isolated from the sample using primers targeting Internal Transcribed Spacer regions; or
iii) the first microorganism is a bacterial species and the second microorganism is a fungal species and DNA isolated from the sample at the first timepoint is divided into a first and second portion and DNA isolated from the sample at the second timepoint is divided into a third and fourth portion, wherein universal bacterial qPCR is conducted on the DNA of the first and third portion and universal fungal qPCR is conducted on the DNA of the second and fourth portion.

In accordance with embodiment 7, the method of any one of embodiments 1-6 is provided wherein the time between said first timepoint and said second timepoint ranges from about 24 hours to 14 days.

In accordance with embodiment 8, the method of any one of embodiments 1-7 is provided wherein the sample, or a portion thereof, is exposed to conditions favorable for growth of viruses present in the sample during the time interval between said first timepoint and said second timepoint, wherein said conditions include the availability of acceptable viral host cells.

In accordance with embodiment 9, the method of any one of embodiments 1-8 is provided wherein the microorganism is an RNA virus and the isolated nucleic acid is RNA that is analyzed by RNA sequencing.

In accordance with embodiment 10, the method of embodiment 9 is provided wherein the RNA of said first and second population of nucleic acids is reverse transcribed using a random hexamer to generate cDNA, and said cDNA is sequenced using next generation sequencing.

In accordance with embodiment 11, the method of any one of embodiments 8-10 is provided wherein the sample, or portion thereof, is further treated by subtractive hybridization to remove host cell RNA and/or DNA prior to conducting qPCR analysis or RNA sequencing of the RNA present in the first and second population of nucleic acids.

In accordance with embodiment 12, the method of any one of embodiments 1-11 is provided wherein the nucleic acid of said first and second population of nucleic acids is DNA, and said DNA is sequenced using next generation sequencing, wherein the generated sequence data is used to identify microorganism species and quantitate the relative amounts of individual microorganism DNAs present in the first and second population of nucleic acids to determine the viability of the identified microorganisms in said sample, optionally wherein the determination of microorganism viability is based on an increase in genome copy number detected at the second timepoint.

### Example 1

### Materials and Methods

### High-Throughput Viability Assay.

Live dead staining of fungi and bacteria have been established and are commercially available. We utilized this method to assess viability of these organisms rapidly after storage in different conditions. For viruses and for complex communities, we established a high-throughput genomic assessment of rapid viability assay for use in evaluation storage and preservation conditions. To make a universal genomic rapid viability assay, we are utilizing the power of RNA sequencing to determine growth. Briefly, RNA was extracted from each fungi, bacteria, virus, or community sample using the Qiagen RNeasy kit. For viruses, subtractive hybridization was utilized to remove the host DNA. Briefly, Host DNA from uninfected cells, was added into the RNA sample at equal DNA: RNA ratio and allowed to hybridize by heating at 95° C. for 2 minutes followed by 65° C. for 5 minutes. RNase H is then added to degrade the host RNA that is hybridized to the DNA. The sample will then be DNase treated to remove the host DNA. Sequencing will then proceed as normal for RNAseq. This procedure was conducted on a T₀ culture and a Tₓ hour culture, where x is the minimum time to observe growth. A spike-in nucleic acid of known copy number was added to both the T₀ and Tₓ samples to serve as a normalization control. The change in mapped k-mer copy number will then be calculated and compared to a culture that had not undergone the preservation conditions being tested with same starting inoculum. This will enable a comparison to the initial sample quantity to determine if there was any loss in viability over storage time.

### Results

### Genomic Viability Assessment Method Development

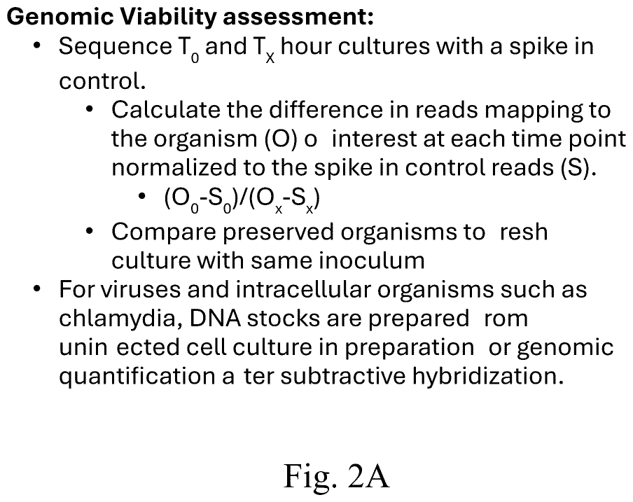

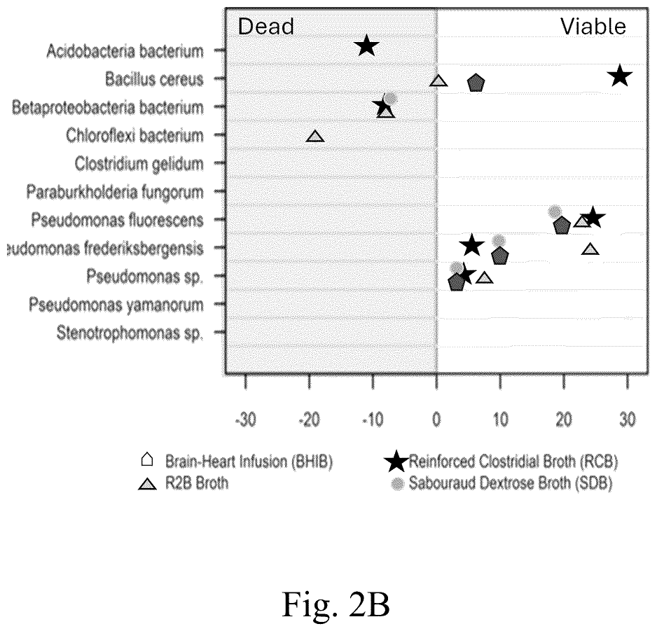

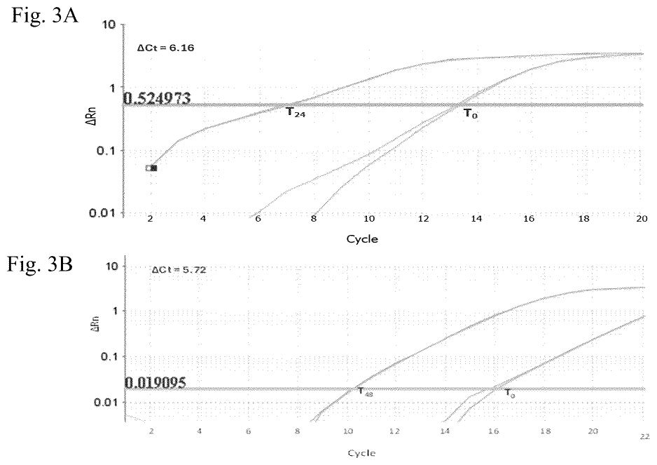

Viability polymerase chain reaction (PCR) methods have previously been developed and demonstrated for detection of viable Legionella pneumophila by reverse transcriptase quantitative PCR (RT-qPCR) and quantitative PCR (qPCR), and viable, virulent Bacillus anthracis in samples from air filters by qPCR using species specific primer sets. Here we are developing universal bacterial RT-qPCR and universal fungal RT-qPCR methods for any individual bacteria, fungi, or total microbial growth within a sample. Briefly, individual bacterial and fungal viability measurements were conducted using RT-qPCR to provide an objective, quantitative measurement of growth by extracting RNA using the RNEasy kit from Qiagen. RNA was extracted from inoculum (T₀) and cultivated samples ranging from 24 hours to 14 days growth (Tₓ, where X=the incubation time). RT-qPCR was then conducted on the T₀ and Tₓ samples using 16S rRNA for bacteria or ITS for fungi as the target. A viability factor was then calculated by ΔCt. Examples of qRT-PCR plots for Bacillus subtilis at 24-hours and Candida albicans at 48 hours are shown in FIG. 2. This method will enable us to calculate fold-change in viability in high throughput when comparing organisms freshly cultured to organisms that had been preserved in our test storage system. To date, the viability factor was calculated for 30 bacteria and 16 fungi. While most bacteria had a positive viability factor at 24 hours, some organisms grow much slower, and therefore had viability factors calculated for when growth was first visible (Table 1).

TABLE 1
Viability factors, cultivation times and 16S copy
numbers for each bacterium tested to date.

| | | 16S Copy | Cultivation | | Viability |
|---|---|---|---|---|---|
| Bacterial Species | Media | Number | Time | | Factor |
| Actinobacillus equuli | TSA/TSB | 6 | 24 | h | 1.78 |
| Actinomyces israelii | BHIA/BHIB | 3 | 24 | h | 3.31 |
| Aeromonas hydrophila | TSA/TSB | 10 | 24 | h | 2.4 |
| Arcanobacterium haemolyticum | SBA/BHIB | 4 | 24 | h | 6.58 |
| Bacillus subtilis | TSA/TSB | 10 | 24 | h | 6.16 |
| Bartonella henselae | Choc/BHIB | 2 | 14 | days | 0.9 |
| Bordetella bronchiseptica | BHIA/BHIB | 3 | 24 | h | 4.36 |
| Campylobacter coli | SBA/BHIB | 3 | 24 | h | 1.45 |
| Campylobacter jejuni subsp. jejuni | SBA/BHIB | 3 | 24 | h | 3.65 |
| Clostridium tetani | SBA/BHIB | 7 | 24 | h | 2.37 |
| Comamonas testosteroni | BHIA/BHIB | 8 | 24 | h | 5.71 |
| Corynebacterium diphtheriae | TSA/TSB | 5 | 24 | h | 4.92 |
| Corynebacterium pseudotuberculosis | BHIA/BHIB | 4 | 24 | h | 2.64 |
| Corynebacterium renale | BHIA/BHIB | 4 | 24 | h | 4.92 |
| Erysipelothrix rhusiopathiae | SBA/BHIB | 6 | 14 | days | 4.75 |
| Escherichia coli | BHIA/BHIB | 7 | 24 | h | 5.88 |
| Klebsiella pneumoniae | BHIA/BHIB | 8 | 24 | h | −15.78 |
| Listeria monocytogenes | BHIA/BHIB | 6 | 24 | h | 4.77 |
| Moraxella catarrhalis | TSA/TSB | 4 | 24 | h | 0.16 |
| Mycobacterium avium | 7H10/7H9 | 1 | 9 | days | 3.03 |
| Nocardia brasiliensis | TSA/TSB | 3 | 7 | days | 7.81 |
| Paenibacillus alvei | TSA/TSB | ~10 | 24 | h | 4.08 |
| Pasteurella multocida subsp. multocida | SBA/BHIB | 6 | 24 | h | 1.58 |
| Phocaeicola vulgatus | SBA/BHIB | 7 | 24 | h | 3.36 |
| Rhodococcus equi | SBA/BHIB | ~4 | 24 | h | 5.3 |
| Salmonella enterica | BHIA/BHIB | 7 | 24 | h | 6.67 |
| Shigella sonnei | BHIA/BHIB | 7 | 24 | h | −8.86 |
| Staphylococcus aureus subsp aureus | BHIA/BHIB | 6 | 24 | h | 8.63 |
| Streptococcus pyogenes | BHIA/BHIB | 6 | 24 | h | 2.24 |
| Trueperella pyogenes | SBA/BHIB | 3 | 9 | days | 6.5 |

All organisms had positive viability factors except for K. pneumoniae and Shigella sonnei.

For samples containing individual fungal species, an ITS viability qPCR was developed for viability assessment, like the 16S viability qPCR for bacteria. For fungi, each species had the viability factor determined at 48 hours, however, this time point was determined to be too early for most species as demonstrated in Table 2.

We will reassess those with viability factors less than 1 at longer cultivation times where we can first visibly see growth. Fungi also tend to grow in clumps or mats and this can hinder accurate nucleic acid extraction, skewing the qRT-PCR results, as not all the nucleic acids from the microbes may be assayed appropriately. Therefore, we will implement the QiaShredder tube to help break apart these mats to enable more accurate results in our optimization of the method.

TABLE 2
Viability factors, media, and cultivation
times for each fungi tested to date.

| | | Cultivation | Viability |
|---|---|---|---|
| Fungal species | Media | Time | Factor |
| Candida albicans | Malt Extract | 48 h | 5.72 |
| | Broth | | |
| Botrytis cinerea | SDA | 48 h | −1.77 |
| Colletotrichum graminicola | OMA/YME | 48 h | −3.40 |
| Cryptococcus neoformans | SDA | 48 h | 0.78 |
| Epidermophyton floccosum | YME | 48 h | −1.23 |
| Magnaporthe poae | OMA | 48 h | −3.78 |
| Melampsora medusae | SDA + filter | 48 h | −0.29 |
| | paper | | |
| Cladosporium cladosporioides | YME | 48 h | −6.14 |
| Mycosphaerella calamagrostidis | YME | 48 h | −3.85 |
| Puccinia canaliculata | OMA | 48 h | −1.44 |
| Sporothrix schenckii | SDA | 48 h | −1.34 |
| Trichophyton rubrum | SDA | 48 h | 0.57 |
| Ustilago maydis | YME | 48 h | 6.41 |
| Cordyceps farinosa | SDA | 48 h | 0.71 |
| Mycosphaerella pinodes* | YME | 48 h | 6.92 |
| Emmonsia ciferrina | Malt Extract | 48 h | 0.90 |
| | Broth | | |

While the universal viability RT-qPCR assays can work for all bacteria and fungi when we identify the correct cultivation times and RNA extraction methods, due to the heterogeneity of viruses, there is no available target for a viral equivalent. Furthermore, in mixed cultures, we will not be able to distinguish which organisms are viable using qPCR, since the method has a single read out for the target instead of a species specific read out. Therefore, a viability sequencing method is used to enable viral viability to be quantified, and enable viability of each member of a community to be determined without a priori knowledge of what organisms are present in the sample. Through our experience in metagenomic and metatranscriptomic analyses of environmental samples, we have demonstrated that viruses are generally poorly represented due to the small genome size of viruses compared to bacteria, fungi, archaea, and eukaryotes. For example, in over 10M reads of sequencing data from COVID-19-positive wastewater, only 4 reads mapped to SARS-COV-2, the virus that causes COVID-19. Therefore, we endeavored to deplete the samples of highly abundant or off target nucleic acids such as ribosomal RNA and host eukaryotic RNA to enable sequencing of the smaller genomes in a mixed sample. Briefly, the method of subtractive hybridization was utilized to remove rRNA from bacteria or fungi following the NEBNext depletion kit for bacteria or human/mouse rat genomes (HMR), respectively. Similarly, subtractive hybridization was utilized on the viral samples to remove any cell line RNA present by using DNA from the cell line as the probe for RNA: DNA hybridization prior to RNase H treatment. RNaseH selectively degrades only RNA hybridized to DNA. Therefore, in a pure RNA sample, only RNA molecules that bind to a DNA probe will be degraded and removed. DNase I is then used to remove the probes, and the RNA sample is depleted of high abundant RNAs such as host RNA or rRNA. Sequencing libraries were then produced using the Kapa RNA HyperPrep kit and sequenced on an Illumina MiSeq Nano flow cell. In our first test of the subtractive hybridization and viability sequencing method, we ran the method on an individual bacteria, fungi, and virus and then sequenced to compare depleted and untreated samples and calculate growth factor from the Tₓ and T₀ time point analysis. To capture the growth effect, we mixed the indexed sample pairs (Tₓ and T₀) prior to library quantification and normalization. This maintains the differential in quantity between the two libraries while ensuring that the right amount of sample is introduced to the flow cell for sequencing.

Sequence alignment of the depleted and undepleted control samples demonstrated that subtractive hybridization reduces the amount of sequence data aligning to ribosomal RNAs for B. subtilis and C. albicans as expected (Table 3). Therefore, this method can be utilized to reduce overabundant sequences to enable detection of smaller genomes in real world or spiked samples which will have a mixture of fungi, bacteria, and viruses. However, due to the high efficiency at which subtractive hybridization removes RNA: DNA hybrids, this method is extremely sensitive to contaminating DNA, as demonstrated by our first round of testing. Due to contaminating DNA present in the RNA samples, very few sequencing reads were mapped to B. subtilis or C. albicans after depletion. However, even without depletion, we were able to produce growth factor ratios validating that sequence data can be used as a measure of viability when comparing cultivation time points. We will repeat this experiment ensuring a DNase treatment occurs prior to depletion with replicates to further validate the method.

TABLE 3
Viability Sequencing Results for B. subtilis,
C. albicans, and Sindbis Virus

| | | | Aligned | | |
|---|---|---|---|---|---|
| | Depletion | Time | Read | Percent | Growth Factor |
| Microbe | (Y/N) | Point | Count | rRNA | (T_{X}/T₀) |
| Bacillus | N | T₀ | 19296 | 89% | 15.67 |
| subtilis | | T₂₄ | 302386 | 86% | |
| | Y | T0 | 6287 | 34% | 0.88 |
| | | T24 | 5532 | 23% | |
| Candida | N | T0 | 56803 | 10% | 5.12 |
| albicans | | T48 | 290868 | 7% | |
| | Y | T0 | 9452 | 35% | 1.78 |
| | | T48 | 16792 | 37% | |
| Sindbis | N | T0 | 3935 | NA* | 56.36 |
| virus | | T12 | 221777 | NA* | |
| | Y | T0 | 15438 | NA* | 0.80 |
| | | T12 | 12346 | NA* | |

The second method tested was viability qPCR using 16S or ITS as the targets. This method is able to determine a growth factor over time for each organism. However, the length of time each organism must cultivate between time points is dependent on the organism, ranging from 12 hours to several weeks, which does not provide much time savings from traditional quantification methods. For example, the fungal ITS viability qPCR needs to be retested with a 7-day time point for most fungi, as 48 hours was not sufficient to generate a growth factor. The third method tested, viability sequencing, also was able to demonstrate an objective, quantifiable measurement of growth over time for viruses, bacteria, and fungi, with the added benefit of being able to be used on microbial communities like we will see in real world samples such as wastewater, soil, and permafrost.

1. A method of identifying viable microorganisms in a sample, said method comprising
i) isolating nucleic acids from a first portion of said sample and determining the concentration of a first microorganism nucleic acid and a second microorganism nucleic acid in said first portion at a first timepoint;
ii) isolating nucleic acids from a second portion of said sample and determining the concentration of said first microorganism nucleic acid and said second microorganism nucleic acid in said second portion of said sample at a second timepoint after the first timepoint, wherein the second portion of said sample is exposed to conditions favorable for growth of microorganisms present in the second portion of said sample during the time interval between said first timepoint and said second timepoint, wherein an increase in the concentration of said first microorganism nucleic acid or said second microorganism nucleic acid at the second timepoint relative to the first timepoint identifies the presence of a viable microbe in the sample.

2. The method of claim 1 wherein said first and second microorganism are organisms independently selected from bacterial, fungal, protists or viral species.

3. The method of claim 1 wherein said first and second microorganism are
i) organisms independently selected from bacterial, fungal and DNA virus species; or
ii) both bacterial species; or
iii) both fungal species;
and the isolated nucleic acid is DNA.

4. The method of claim 1 wherein said first and second microorganism are RNA viruses and the isolated nucleic acid is RNA.

5. The method of claim 4, wherein the isolated nucleic acid is RNA, and the concentration of said first microorganism nucleic acid and said second microorganism nucleic acid is measure by conducting a reverse transcriptase quantitative polymerase chain reaction (RT-qPCR).

6. The method of claim 3 wherein an equal volume of extracted nucleic acids isolated from said first timepoint sample and said second timepoint sample are analyzed, further wherein:
i) the first microorganism and the second microorganism are each bacterial species and a universal bacterial qPCR is conducted on DNA isolated from the first timepoint sample and the second timepoint sample, using primers targeting 16S rRNA encoding sequences; or
ii) the first microorganism and the second microorganism are each fungal species and a universal fungal qPCR is conducted on DNA isolated from the first timepoint sample and the second timepoint sample using primers targeting Internal Transcribed Spacer regions; or
iii) the first microorganism is a bacterial species and the second microorganism is a fungal species and DNA isolated from the first timepoint sample is divided into a first and second nucleic acid portion and DNA isolated from the second timepoint sample is divided into a third and fourth nucleic acid portion, wherein universal bacterial qPCR is conducted on the DNA of the first and third nucleic acid portion and universal fungal qPCR is conducted on the DNA of the second and fourth nucleic acid portion.

7. The method of claim 1 wherein the time between said first timepoint and said second timepoint ranges from about 24 hours to 14 days.

8. A method of detecting viable microorganisms in a sample, said method comprising
isolating a first population of nucleic acids from a first portion of said sample;
exposing a second portion of said sample to conditions favorable to the growth and replication of microorganisms, for a predetermined length of time;
isolating a second population of nucleic acids from said second portion of said sample after said predetermined length of time;
sequencing nucleic acids from a equivalent volume of said first and second populations of nucleic acids; and
conducting a quantitative comparison of the sequenced nucleic acids of the first and second populations of nucleic acids wherein an increase in the number of the sequenced nucleic acids in the second population of nucleic acids relative to the sequenced nucleic acids in the first population of nucleic acids indicates the presence of viable microorganisms in the sample.

9. The method of claim 1 wherein a portion of said sample is exposed to conditions favorable for growth of viruses present in the sample during the time interval between said first timepoint and said second timepoint, wherein said conditions include the availability of acceptable viral host cells.

10. The method of claim 9, wherein said microorganism is an RNA virus and the isolated nucleic acid is RNA that is analyzed by RNA sequencing, using equivalent volumes of the nucleic acid extracts prepared from the first and second timepoint samples.

11. The method of claim 10 wherein the RNA of said first and second population of nucleic acids is reverse transcribed using a random hexamer to generate cDNA, and said cDNA is sequenced using next generation sequencing.

12. The method of claim 11, wherein the sample, or portion thereof, is further treated by subtractive hybridization to remove host cell RNA and/or DNA prior to conducting qPCR analysis or RNA sequencing of the RNA present in the first and second population of nucleic acids.

13. The method of claim 1 wherein the nucleic acid of said first and second population of nucleic acids is DNA, and said DNA is sequenced using next generation sequencing, and the generated sequence data is used to identify microorganism species and quantitate the relative amounts of individual microorganism DNAs present in the first and second population of nucleic acids to determine the viable organisms of the identified microorganisms in said sample.

14. The method of claim 13 wherein the determination of microorganism viability is based on an increase in genome copy number detected at the second timepoint.

15. A method of detecting viable microorganisms in a sample comprising:
i) preparing a time zero sample (t0) and separating the time zero sample (t0) into a first portion time zero sample (t0) and a second portion time zero sample (t0);
ii) extracting nucleic acids from said first portion time zero sample (t0);
iii) quantitating said nucleic acids extracted from said first portion time zero sample (t0);
iv) culturing said second portion time zero sample (t0) under conditions favorable for growth of microbes for a time interval to produce a time x (tx) sample;
v) extracting nucleic acids from said time x sample (tx);
quantitating the nucleic acids extracted from said time x sample (tx) relative to the quantitation of the nucleic acids in said time zero sample (t0) to identify the presence of viable microbes.
